(19) United States
(12) Patent Application Publication
(10) Pub. No.: US 2013/0190518 A1
(43) Pub. Date: Jul. 25, 2013

(54) TERPENOID DERIVATIVES OBTAINED FROM TERPENOIDS STEMING FROM RENEWABLE SOURCES

(71) Applicants: Marc Mauduit, Vitre (FR); Frédéric Caijo, Thorigne-Fouillard (FR); Christophe Crévisy, Pont-Pean (FR)

(72) Inventors: Marc Mauduit, Vitre (FR); Frédéric Caijo, Thorigne-Fouillard (FR); Christophe Crévisy, Pont-Pean (FR)

(73) Assignee: Ecole Nationale Superieure de Chimie de Rennes, Rennes (FR)

(21) Appl. No.: 13/809,313

(22) PCT Filed: Jul. 11, 2011
(86) PCT No.: PCT/EP2011/061775

§ 371 Date: Mar. 19, 2013

Related U.S. Application Data
(60) Provisional application No. 61/362,991, filed on Jul. 9, 2010.

(51) Int. Cl. C07C 67/347; C07C 45/71; C07C 69/593; C07C 47/263; C07C 33/035; C07C 69/732; C07C 29/46

(52) U.S. Cl. CPC C07C 67/347; C07C 69/732; C07C 45/71; C07C 29/46; C07C 47/263; C07C 33/035; C07C 69/593
USPC 554/121; 554/223; 568/460; 568/904; 568/496; 568/909.5

(57) ABSTRACT

The present invention relates to a process for preparing a terpenoid derivative, the process comprising a metathesis of an olefin and a terpenoid, and to terpenoid derivatives prepared with said process.

### BACKGROUND

Owing to both the decrease of the oil stocks and the rise of their price, and environmental aspects such as green house effect, research attention has recently been focused on the use of renewable resources isolated from agro-resources to produce various types of organic compounds, such as for example raw materials, intermediates, fine chemicals, organic polymers, and solvents (Monomers, polymers and composites from renewable resources, M. N. Belgacem and A. Gandini Eds; Elsevier, Amsterdam, 2008; A. Corma, S. Iborra, A. Velty, Chem. Rev. 2007, 107, 2411-2502.).

Among the products that can be isolated from agro-resources, such as vegetable oils and sugars, terpenes and terpenoids appear particularly attractive. Terpenoids are a class of compounds formally assembled from terpene building blocks.

The term “terpenes” is generally used to indicate compounds derived from five-carbon isoprene units, while the term “terpenoids” is generally used to indicate modified “terpenes”, such as for example terpene oxygen-containing compounds such as alcohols, aldehydes or ketones. If not otherwise indicated, in the present disclosure and in the following claims the terms “terpenes” and “terpenic compounds” will include also “terpenoids”, respectively “terpenoid compounds”. The terms “terpenoid derivative” is generally used to indicate compounds derived from terpenic compounds.

Some terpenes, mainly the most expensive ones, are used directly but many transformation processes have been necessary to transform the cheapest compounds, such as for example pinene, limonene, citral, into high added value products such as fragrances (Monomers, polymers and composites from renewable resources, M. N. Belgacem and A. Gandini Eds; Elsevier, Amsterdam, 2008; A. Corma, S. Iborra, A. Velty, Chem. Rev. 2007, 107, 2411-2502).

The prior art processes not only require an excessive number of steps, but are not sufficiently eco-compatible.

To be more “eco-compatible”, the chemical processes dedicated to the conversion of raw materials obtained from renewable resources must use environmentally benign reactions. Catalyzed reactions are particularly suitable to reach that aim. Ruthenium-based catalysts are known for their use as olefin metathesis catalysts in olefin metathesis of terpenic compounds, as disclosed in (a) Vieille-Petit, L.; Clavier, H.; Linden, A.; Blumentritt, S.; Nolan, S. P.; Dorta, R. Organometallics, 2010, 29, 775, (b) Monfette, S.; Camm, K. D.; Gorelsky, S. I.; Fogg, D. E. Organometallics 2009, 28, 944 and (c) Conrad, J. C.; Parnas, H. H.; Snelgrove, J. L.; Fogg, D. E. J. Am. Chem. Soc. 2005, 127, 11882.

(d) Hoye, T. R.; Zhao, H. Org. Lett. 1999, 1, 1123 and (e) Mathers, R. T.; McMahon, K. C.; Damodaran, K.; Retarides, C. J.; Kelley, D. J. Macromolecules 2006, 39, 8982 disclose the use of ruthenium-based catalysts for the ring-opening metathesis of D-limonene and the ring-closing metathesis of, for example, citronellene.

Consequently, there exists the need for a simple and eco-compatible process for the transformation of terpenoids obtained from renewable resources.

### SUMMARY OF THE INVENTION

According to a first aspect, the present disclosure relates to a process for transforming a terpenoid into a terpenoid derivative, the process comprising at least one metathesis of an olefin and the terpenoid, wherein the terpenoid has the following general formula:

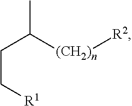

wherein R¹ is

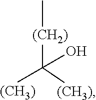

n is 0 and R² is

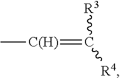

or
   R¹ is or

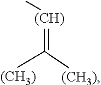

n is 0 and R² is

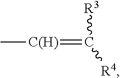

or
R¹ is or

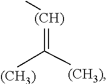

n is 1 and R² is —C(H)═O or —C(H₂)—OAc, and
wherein R³ and R⁴ are the same or different and are each independently hydrogen or alkyl,
wherein the olefin has the following general formula:

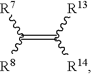

wherein R⁷, R⁸, R¹³ and R¹⁴ are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine;
wherein when n=1, R⁷ and R⁸ are not both —CH₃; and
wherein when n=0, R⁷ and R⁸ together are different from R³ and R⁴ together.

According to an embodiment of the present invention, the process comprises a first metathesis of a first olefin as described above and a terpenoid as described above to prepare a first terpenoid derivative and a second metathesis of a second olefin and the first terpenoid derivative, wherein the second olefin has the following general formula:

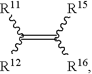

wherein R¹¹, R¹², R¹⁵ and R¹⁶ are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine;
wherein R¹¹ and R¹² are not both —CH₃; and
wherein R¹¹ and R¹² together are different from R³ and R⁴ together.

According to an embodiment, the terpenoid has only one double bond.

According to an embodiment, the terpenoid has a leaving group which can be eliminated by an elimination reaction, such as for example a hydroxyl group. In this case, the process may further comprise an elimination reaction, which is dehydration if the leaving group is a hydroxyl group.

According to an embodiment, the terpenoid has at least two double bonds. In this case, in a preferred embodiment, the process further comprises oxidizing at least one allylic carbon of the terpenoid prior to the olefin metathesis.

According to an embodiment, the terpenoid has two double bonds and the process further comprises protecting one of the two double bonds with a leaving group, for example with a hydroxyl group.

According to an embodiment, the olefin metathesis is a olefin cross-metathesis. In a preferred embodiment, the olefin cross-metathesis is a catalyzed olefin cross-metathesis, for example with a ruthenium Hoveyda type catalyst.

According to a second aspect, the present disclosure relates to novel terpenoid derivatives having the following general formula:

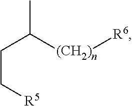

wherein R⁵ is

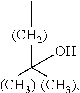

n is 0 and R⁶ is

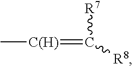

or
   R⁵ is

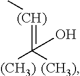

n is 0 and R⁶ is

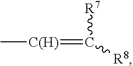

or
R⁵ is

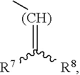

n is 0 and R⁶ is

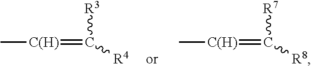

or
R⁵ is

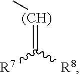

n is 1 and R⁶ is —C(H)═O or —C(H₂)—OAc,
wherein R³ and R⁴ are as described above,
wherein R⁷ and R⁸ are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine, and
wherein when R⁵ is

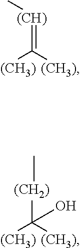

R⁷ and R⁸ are not both hydrogen, and
wherein when R⁵ is

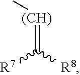

R⁷ and R⁸ are not both —CH₃.

According to a third aspect, the present disclosure relates to novel terpenoid derivatives having the following general formula:

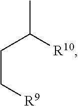

wherein R⁹ is

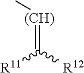

and R¹⁰ is

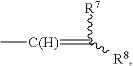

or
   R⁹ is

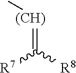

and R¹⁰ is

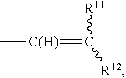

wherein R⁷ and R⁸ are as described above,
wherein R¹¹ and R¹² are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine,
wherein when R⁹ is

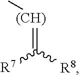

and when R¹¹ and R¹² are both hydrogen, R⁷ and R⁸ are not both —CH₃, and
wherein when R⁹ is

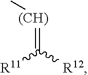

and when R⁷ and R⁸ are both hydrogen, R¹¹ and R¹² are not both —CH₃.

According to an embodiment, R⁷, R⁸, R¹¹, R¹² are the same or different and are each independently hydrogen, alkyl, for example a lower alkyl, aryl, ketone, ester, ether, amide, or sulfonamide.

According to an embodiment of the present disclosure, the terpenoid derivative is obtained by the process according to the first aspect of the present invention.

According to a fourth aspect, the present disclosure relates to the use of ruthenium Hoveyda type catalysts for the catalyzed cross-metathesis of a terpenoid with an olefin.

### DETAILED DESCRIPTION OF EMBODIMENTS

Terpenoids, catalysts, terpenoid derivatives and processes for the transformation of terpenoids in terpenoid derivatives are described in the following.

As most terpenic compounds contain one or more carbon-carbon double bond, olefin metathesis, which in oleochemistry has been considered as a versatile tool for thirty years, is potentially a tool of choice to convert them into valuable products with a high selectivity.

The process comprises the catalyzed transformation of terpenoids by at least one olefin metathesis reaction.

According to an embodiment of the present invention, the terpenoids that may be transformed by olefin metathesis may be any compound having the following general formula (I):

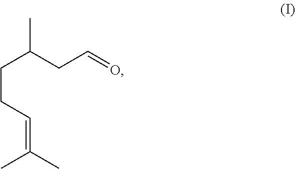

or the following general formula (II):

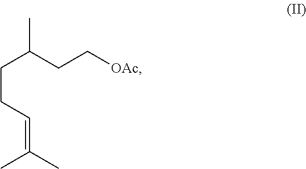

or the following general formula (III):

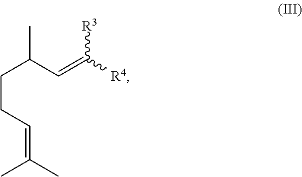

or the following general formula (IV):

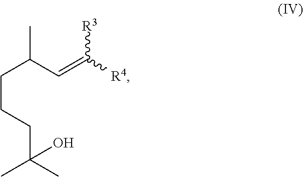

wherein R³, R⁴ are the same or different and each may be independently hydrogen or alkyl.

According to an embodiment of the present invention, the process may comprise one olefin metathesis with an olefin and a terpenoid as described above, the olefin having the following general formula:

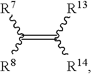

wherein R⁷, R⁸, R¹³ and R¹⁴ are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine;
wherein when n=1, R⁷ and R⁸ are not both —CH₃; and
wherein when n=0, R⁷ and R⁸ together are different from R³ and R⁴ together.

According to an embodiment of the invention, the process may comprise a first olefin metathesis with a first olefin as described above and a terpenoid as described above to prepare a first terpenoid derivative and a second olefin metathesis of a second olefin and the first terpenoid derivative to prepare a second terpenoid derivative. The second olefin may have the following general formula:

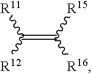

wherein R¹¹, R¹², R¹⁵ and R¹⁶ are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine;
wherein R¹¹ and R¹² are not both —CH₃; and
wherein R¹¹ and R¹² together are different from R³ and R⁴ together.

According to an embodiment, the terpenoids are monoterpenoids.

The process of the present invention comprises a reaction based on olefin metathesis of terpenoids, for example olefin cross-metathesis.

When the process comprises reacting terpenoids comprising one double bond, the terpenoids can be reacted as such.

When the process comprises reacting terpenoids comprising two double bonds, one of the two double bonds is preferably oxidized. The oxidation may introduce for example a hydroxy, aldehyde, ketone or epoxide group.

When the process comprises reacting terpenoids comprising two double bonds, one of the two double bonds is preferably protected with a leaving group, for example with a hydroxyl group or any other group which can be for example eliminated by an elimination reaction. The leaving group may be for example a hydroxyl group. In this case, the elimination reaction is dehydration.

When the process comprises reacting terpenoids comprising more than two double bonds, the double bonds exceeding one are preferably protected with respective leaving groups.

According to an embodiment, R¹ is

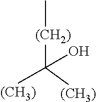

and the process further comprises a dehydration reaction after the olefin metathesis.

According to an embodiment, the process is carried out in the presence of an olefin metathesis catalyst, for example an organometallic catalyst.

According to a preferred embodiment of the present invention, the olefin metathesis catalyst is a Hoveyda type catalyst, for example a ruthenium Hoveyda type catalyst.

Ruthenium Hoveyda type catalysts, containing an aminocarbonyl function linked to the benzylidene ligand, were used.

According to a preferred embodiment of the present invention, the Ruthenium Hoveyda type catalysts may have the following general formula:

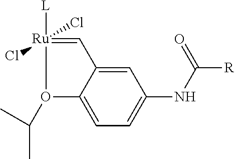

wherein L is SIMes or SIPr and R is CF₃, CO₂Et, OiBu, C₆F₅ or C₁₅H₃₁.

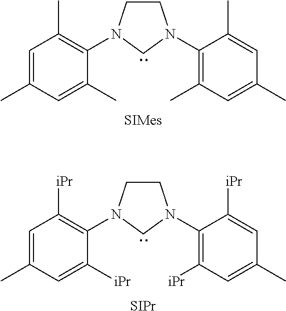

According to another preferred embodiment of the present invention, the Ruthenium Hoveyda type catalysts may have the following general formula:

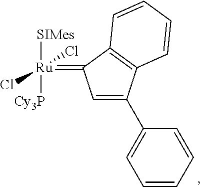

or the general formula:

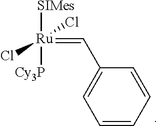

The novel terpenoid derivatives according to the present invention may have the general formula (V):

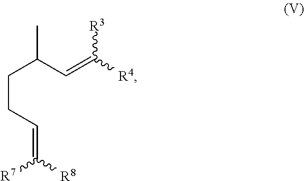

or the general formula (VI):

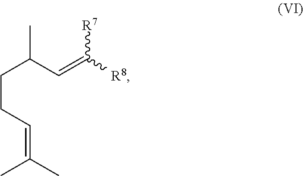

or the general formula (VII):

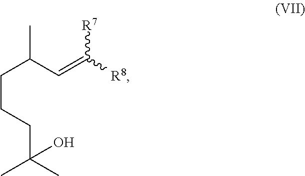

or the general formula (VIII):

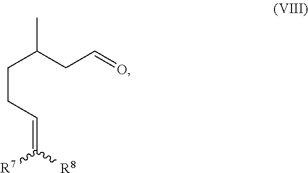

or the general formula (IX):

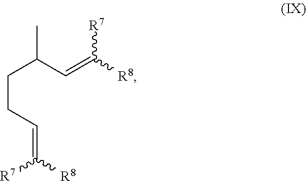

or the general formula (X):

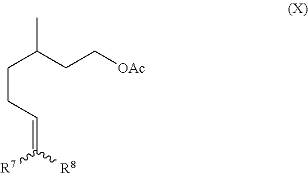

wherein R³ and R⁴ are as described above,
wherein R⁷ and R⁸ are the same or different and each may be independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine,
wherein when the terpenoid derivative has the general formula (VI), (VII) or (IX), R⁷ and R⁸ are not both hydrogen, and
wherein when the terpenoid derivative has the general formula (V), (VIII) or (X), R⁷ and R⁸ are not both —CH₃.

According to an embodiment, terpenoid derivatives having the general formulae V, VI, VIII or IX, as described above, may then be transformed to second terpenoid derivatives by a second olefin cross-metathesis of the terpenoid derivative and the second olefin.

Further novel terpenoid derivatives according to the present invention may have the following general formula (XI):

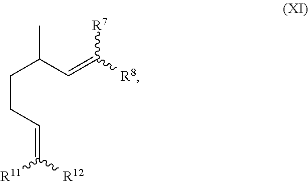

or the general formula (XII):

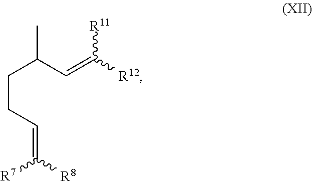

wherein R⁷ and R⁸ are as described above,
wherein R¹¹ and R¹² are the same or different and each may be independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine,
wherein when the terpenoid derivative has the general formula XII and when R¹¹ and R¹² are both hydrogen, R⁷ and R⁸ are not both —CH₃, and
wherein the terpenoid derivative has the general formula XI and when R⁷ and R⁸ are both hydrogen, R¹¹ and R¹² are not both —CH₃.

According to an embodiment, R⁷, R⁸, R¹¹ and R¹² are the same or different and each may be independently hydrogen, alkyl, for example a lower alkyl, aryl, ketone, ester, ether, amide, or sulfonamide.

Each of R⁷, R⁸, R¹¹ and R¹² may optionally be substituted.

As used herein, the term “alkyl” refers to an aliphatic group that is branched or unbranched and is a saturated hydrocarbon group of 1 to 24 carbon atoms, such as methyl, ethyl, n-propyl, isopropyl, n-butyl, isobutyl, s-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms. The terms “halogenated alkyl” or “haloalkyl group” refer to an alkyl group as defined above with one or more hydrogen atoms present on these groups substituted with a halogen (F, Cl, Br, I). Exemplary haloalkyl groups include perhaloalkyl groups, wherein all of the hydrogen atoms present on the group have been replaced with a halogen, for example perfluoromethyl refers to the group —CF₃. The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom in the ring such as, but not limited to, nitrogen, oxygen, sulfur, or phosphorous. In contrast with heterocycloalkyl groups, the term “alicyclic” refers to a group that is both aliphatic and cyclic. Such groups contain one or more saturated or unsaturated all-carbon rings, which are not aromatic. Alkyl groups, including cycloalkyl groups and alicyclic groups optionally may be substituted. The nature of the substituents can vary broadly. Typical substituent groups useful for substituting alkyl groups in the presently disclosed compounds include halo, fluoro, chloro, alkyl, alkylthio, alkoxy, alkoxycarbonyl, arylalkyloxycarbonyl, aryloxycarbonyl, cycloheteroalkyl, carbamoyl, haloalkyl, dialkylamino, sulfamoyl groups and substituted versions thereof.

The term “alkenyl” refers to a hydrocarbon group of 2 to 24 carbon atoms and structural formula containing at least one carbon-carbon double bond.

The term “alkynyl” refers to a hydrocarbon group of 2 to 24 carbon atoms and a structural formula containing at least one carbon-carbon triple bond. The term “aliphatic” refers to moieties including alkyl, alkenyl, alkynyl, halogenated alkyl and cycloalkyl groups as described above. A “lower aliphatic” group is a branched or unbranched aliphatic group having from 1 to 10 carbon atoms.

The term “amine” or “amino” refers to a group of the formula —NR′R″, where R′ and R″ may be the same or different and independently are hydrogen or an alkyl, alkenyl, alkynyl, aryl, aralkyl, cycloalkyl, halogenated alkyl, or heterocycloalkyl group described above. The term “amide” refers to a group represented by the formula —C(O)NR′R″, where R′ and R″ independently can be a hydrogen, alkyl, alkenyl, alkynyl, aryl, aralkyl, cycloalkyl, halogenated alkyl, or heterocycloalkyl group described above.

The term “aryl” refers to any carbon-based aromatic group including, but not limited to, benzyl, naphthyl, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted. The term “alkyl amino” refers to alkyl groups as defined above where at least one hydrogen atom is replaced with an amino group.

The term “aralkyl” refers to an aryl group having an alkyl group, as defined above, attached to the aryl group. An example of an aralkyl group is a benzyl group.

Optionally substituted groups, such as “substituted alkyl,” refer to groups, such as an alkyl group, having from 1-5 substituents, typically from 1-3 substituents, selected from alkoxy, optionally substituted alkoxy, acyl, acylamino, acyloxy, amino, aminoacyl, aminoacyloxy, aryl, carboxyalkyl, optionally substituted cycloalkyl, optionally substituted cycloalkenyl, optionally substituted heteroaryl, optionally substituted heterocyclyl, hydroxy, thiol and thioalkoxy.

The term “carbonyl” refers to a radical of the formula —C(O)—. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR′ or —CHO where R′ is an aliphatic, heteroaliphatic, alkyl, heteroalkyl, hydroxyl, or a secondary, tertiary, or quaternary amine.

The term “carboxyl” refers to a —COOH radical. Substituted carboxyl refers to —COOR′ where R′ is aliphatic, heteroaliphatic, alkyl, heteroalkyl, aralkyl, aryl or the like. The term “derivative” refers to compound or portion of a compound that is derived from or is theoretically derivable from a parent compound.

The term “hydroxyl” refers to a moiety represented by the formula —OH. The term “alkoxy group” is represented by the formula —OR′, wherein R′ can be an alkyl group, optionally substituted with an alkenyl, alkynyl, aryl, aralkyl, cycloalkyl, halogenated alkyl, or heterocycloalkyl group as described above.

The term hydroxyalkyl refers to an alkyl group that has at least one hydrogen atom substituted with a hydroxyl group. The term “alkoxyalkyl group” is defined as an alkyl group that has at least one hydrogen atom substituted with an alkoxy group described above. Where applicable, the alkyl portion of a hydroxyalkyl group or an alkoxyalkyl group can be substituted with aryl, optionally substituted heteroaryl, aralkyl, halogen, hydroxy, alkoxy, carboxyalkyl, optionally substituted cycloalkyl, optionally substituted cycloalkenyl and/or optionally substituted heterocyclyl moieties.

Valuable terpenoid intermediates and products were produced by using catalysts with olefin cross-metathesis substrates starting from terpenes or derivatives thereof.

### EXAMPLES

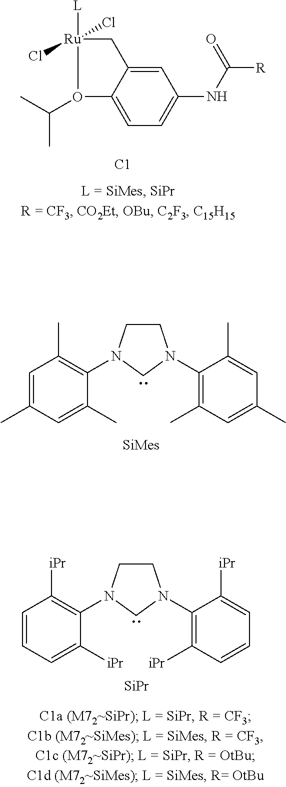

General informations: 1H (400 MHz) and 13C (100 MHz) NMR spectra were recorded on a Bruker ARX400 spectrometer with complete proton decoupling for nucleus other than 1H. Chemical shifts are reported in ppm with the solvent resonance as the internal standard (CDCl3, δ 7.26 ppm, 13C: δ 77.00 ppm). Data are reported as follows: chemical shift δ in ppm, multiplicity (s=singlet, d=doublet, t=triplet, q=quadruplet, hept=heptuplet, m=multiplet), coupling constants (Hz), integration and attribution.

All non-aqueous reactions were performed under an argon atmosphere. HPLC grade AcOEt was used. n-Butyl acrylate, acrolein, crotonaldehyde were distilled before use. All others chemical reagents and solvents were obtained from commercial sources and used without further purification. Olefin metathesis catalysts C1, C2 and C3 are commercially available complexes.

General Procedure for the Olefin Cross-Metathesis

The catalyst was introduced in a round bottom flask under argon. The solvent and the two olefinic compounds were added. The solution was carefully degassed (3 vacuum/argon cycles) then was heated and stirred the required period of time. When the reaction was completed, the solvent was removed under vacuum and the residue was purified by flash chromatography (cyclohexane/ethyl acetate).

### Example 1

The reactivity of different terpenoids compounds, using different olefins in the presence of catalyst C1a according to Scheme 1, was investigated.

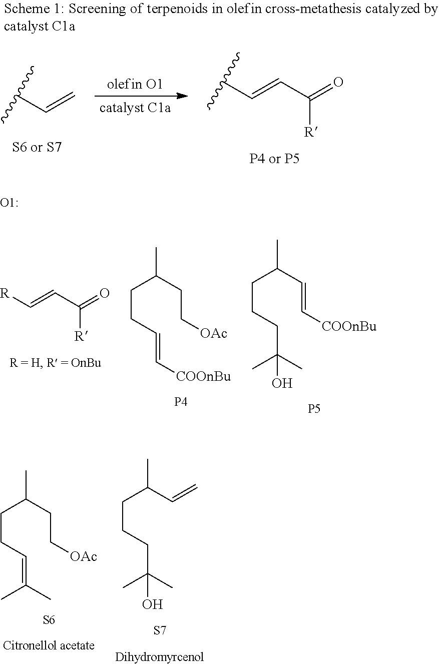

The reaction was run in ethylacetate solvent and the results are summarized in Table 1.

TABLE 1
Screening of terpenoid compounds S6-S7 in olefin cross-metathesis

| | Olefin | | | |
|---|---|---|---|---|
| Substrate | (n eq.) | Catalyst loading | Conditions | Product (yield) |
| S6 | O1 (2) | 1 mol % | 80° C., 1 h, | P4 (43%) |
| | | | AcOEt (0.25 M) | |
| S7 | O1 (2) | 1 mol % | 60° C., 17 h, | P5 (71%) |
| | | | AcOEt (1 mL/1 | |
| | | | mmole S7) | |

Terpenoid compounds having only one double bond were reacted. When citronellol acetate S6 was reacted with n-butyl acrylate (2 eq.) O1 in AcOEt in the presence of 1 mol % of catalyst C1a, the expected product P4 was obtained in 43% isolated yield.

Terpenoid compounds having two double bonds were reacted. The second double bond was masked, for instance as the hydrated form. The reactivity of dihydromyrcenol S7 was evaluated. S7 can be considered as a derivative of citronellene where one double bond is masked as a hydroxyl group. The double bond could be regenerated later from the alcohol through a simple elimination reaction. When S7 was reacted 17 h at 60° C. with n-butyl acrylate (2 eq.) O1 and in the presence of 1 mol % of catalyst C1a, the expected olefin cross-metathesis product P5 was obtained in an isolated yield of 71%.

### Example 2

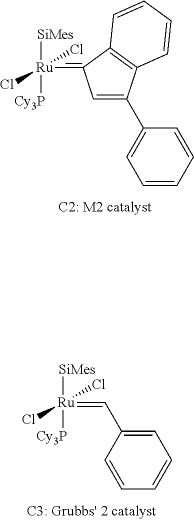

Four catalysts “Hoveyda type” boomerang Ruthenium catalysts C1a-d containing an aminocarbonyl function was evaluated in the model reaction of dihydromyrcenol S7 and n-butyl acrylate O1 (Table 2). Two supplementary commercial catalysts, M2 catalyst C2, available from Umicore, and Grubbs' 2 catalyst C3 were also tested. 1 mol % catalyst was used and the reagents were heated at 60° C. in ethylacetate during 17 h. While catalysts C1a-d and C2 showed similar behaviors (Table 2), affording the expected olefin cross-metathesis product P5 with good yields (63-73%), a bad result was observed with Grubbs'2 catalyst C3 which afforded P5 in low yield (25%).

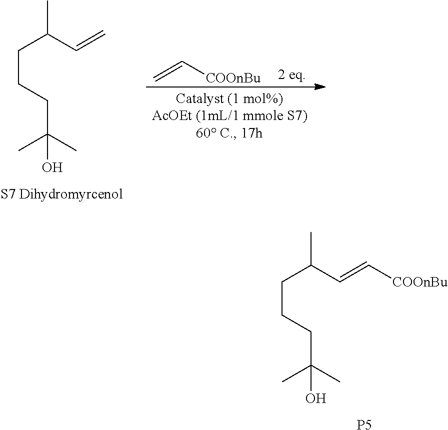

TABLE 2
Evaluation of catalysts C1a-d and C2-C3 in the olefin
cross-metathesis of S7 and n-butyl acrylate O1

| Catalyst | P5 isolated yield (%) |
|---|---|
| C1a | 71 |
| C1b | 63 |
| C1c | 73 |
| C1d | 67 |
| C2 | 68 |
| C3 | 25 |

### Example 3

The reactivity of dihydromyrcenol S7 was then evaluated towards other olefins, using the catalyst C1a in ethylacetate as the solvent (Scheme 2, Table 3).

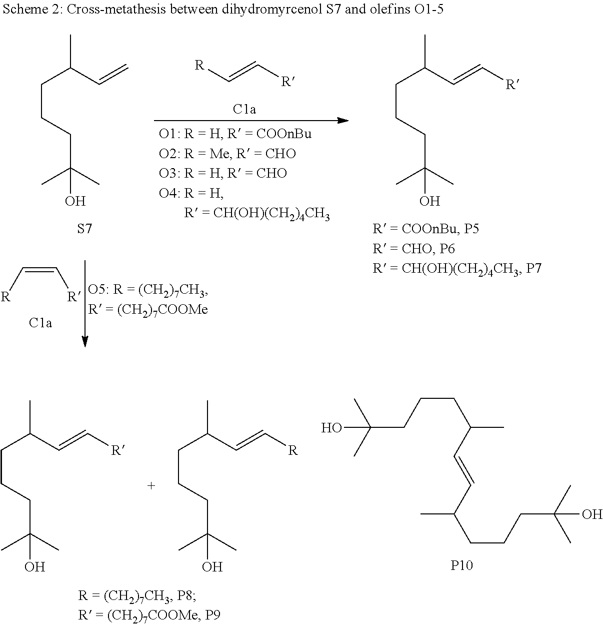

TABLE 3
Cross-metathesis between dihydromyrcenol S7 and olefins O1-5

| | Catalyst | | | |
|---|---|---|---|---|
| Olefin (n eq.) | loading (mol %) | Conditions | | Product (yield) |
| O1(2) | 1 | 60° C., | 18 h | P5 (75%) |
| O1(2) | 0.5 | 60° C., | 17 h | P5 (47%) |
| O1(2) | 0.2 | 60° C., | 17 h | P5 (28%) |
| O2(1.2) | 1 | 60° C., | 23 h | P6 (53%)^{a} |
| O3(1) | 0.5 | 60° C., | 16 h | P6 (80%)^{b} |
| O4(1) | 1 | 50° C., | 24 h | P7 (43%) |
| O5(1.5) | 2 | 80° C., | 3 h | P8 (61%)^{c} |
| | | | | P9 (71%)^{d} |
| S7 | 1 | 80° C., | 3 h | P10 (82%) |

^{a}Isolated as a (E)/(Z) 95/5 mixture.
^{b}Isolated as a (E)/(Z) 94/6 mixture.
^{c}Isolated as a (E)/(Z) 87/13 mixture.
^{d}Isolated as a (E)/(Z) 86/14 mixture.

The first olefin studied was n-butyl acrylate O1. After 18 h at 60° C. in the presence of 1 mol % of catalyst, P5 could be isolated in 75% yield. The decrease of the loading to 0.5 mol % caused a significant drop of the yield (47%). A further decrease of the catalyst loading (0.2 mol %) caused a further drop of the efficiency of the reaction; 28% yield was obtained after 17 h of reaction.

A similar behavior was observed with crotonaldehyde O2 since, in the presence of 1 mol % of C1a, P6 was isolated in 53% yield after 23 h at 60° C. A far better result was observed with acrolein O3 since P6 could also be isolated with a higher yield (80%) although only 0.5 mol % catalyst was used. A lower yield (43%) was obtained in product P7 when 1-octen-3-ol O4 was used as the olefin.

The reaction of dihydromyrcenol S7 and methyl oleate O5 in the presence of 2 mol % of C1a afforded the two expected products in good isolated yields (61% for P8 and 71% for P9). It must be noted that competitive isomerisation of the double bond is likely to occur in this case as the presence of a small amount (<10%) of an impurity having one CH₂ group missing (M-14) could be detected in HRMS experiments of P8 and P9.

When dihydromyrcenol S7 was reacted at 80° C. in the presence of 1 mol % of catalyst and in the absence of a second olefin, self metathesis occurred which afforded P10 as a mixture of diastereoisomers in a good yield (82%).

### Example 4

Finally, the possibility to regenerate a double bond through the elimination of the alcohol group was checked (Scheme 3).

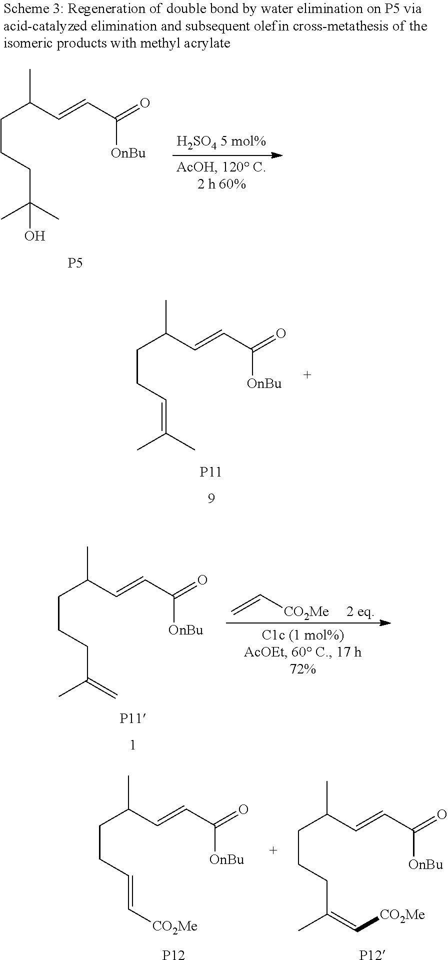

To compound P5 (600 mg, 2.34 mmole) was added a 5 mol % solution of sulphuric acid in AcOH (6 μl/600 μL). The solution was heated 2 h at 120° C. The mixture was then diluted in AcOEt and the organic phase was washed with a saturated solution of NaHCO₃ then dried (MgSO₄). The solvent was removed under vacuum and the residue was purified by flash chromatography (Cyclohexane/AcOEt 90/10) to give the mixture of P11 and P11′ in a 9/1 ratio as a colourless oil (334 mg) in rather good yield (60%).

A final olefin cross-metathesis between the regenerated double bond of P11 and methyl acrylate was then undertaken in order to validate the strategy suggested to overcome the selectivity problem. Thus, the P11/P11′ mixture was reacted 17 h at 60° C. with methyl acrylate in the presence of 1 mol % of catalyst C1c, which afforded the expected products (P12/P12′: ˜9/1) in a rather good yield (72%). This result demonstrates that the difficulty arising from the presence of two double bonds in many terpenes (as citronellene) can be overcome by the protection of one of these double bonds as an alcohol.

To conclude, the Applicant has shown that by using masked derivatives such as dihydromyrcenol where one double bond is protected as the hydrated form, high selectivity can be obtained in the olefin cross-metathesis of terpenoid compounds having more than one double bond. The cross-metathesis between dihydromyrcenol and various olefins was successfully proven, showing that olefin cross-metathesis is suitable for the synthesis of valuable synthetic intermediates from renewable terpenoid feedstocks.

NMR Data

Compound P4: ¹H RMN δ (ppm)=0.92 (d, J=6.6 Hz, 3H, CH—CH₃); 0.93 (t, J=7.4 Hz, 3H, CH₂CH₃); 1.23-1.73 (m, 9H, 4CH₂ and CH); 2.03 (s, 3H, CH₃CO); 2.13-2.28 (m, 2H, CH₂CH═); 4.03-4.16 (m, 4H, 2CH₂O); 5.81 (dt, J=15.6, 1.5 Hz, 1H, CH═CHCO); 6.94 (dt, J=15.6, 6.8 Hz, 1H, CH═CHCO). ¹³C RMN δ (ppm)=13.7, 19.1 (2), 21.0, 29.3, 29.5, 30.7, 35.0, 35.2, 62.6, 64.1, 121.4, 148.9, 166.7, 171.1

Compound P5: ¹H RMN δ (ppm): 0.93 (t, J=7.2 Hz, 3H, CH₂—CH₃); 1.05 (d, J=6.4 Hz, 3H, CH—CH₃); 1.19 (s, 6H, C(OH)(CH₃)₂); 1.31-1.67 (m, 10H, 5CH₂); 2.27-2.37 (m, 1H, CH), 4.13 (t, J=6.8 Hz, 2H, CH₂O); 5.77 (dd, J=15.6, 1.0 Hz, 1H, CH═CHCO); 6.85 (dd, J=15.6, 8.0 Hz, 1H, CH═CHCO). ¹³C RMN, δ (ppm): 13.7; 19.2; 19.4; 21.9; 29.3; 30.7; 36.4; 36.5; 43.8; 64.1; 70.9; 119.7; 154.4; 167.0. HRMS (ESI) calcd for C₁₅H₂₈O₃Na: 279.1936; found: 279.1928 (3 ppm).

Compound P6 (E isomer): ¹H RMN δ (ppm): 1.10 (d, J=6.8 Hz, 3H, CH—CH₃); 1.20 (s, 6H, C(OH)(CH₃)₂); 1.32-1.48 (m, 6H, 3CH₂); 2.40-2.51 (m, 1H, CH); 6.08 (ddd, J=15.6, 7.6,1.2 Hz, 1H, CHCHO); 6.74 (dd, J=15.6, 7.6 Hz, 1H, CH═CHCHO); 9.50 (d, J=7.6 Hz, 1H, CHO). ¹³C RMN δ (ppm): 19.1; 21.9; 29.2; 29.3; 36.3; 37.0; 43.7; 70.8; 131.3; 163.9; 194.3. HRMS (ESI) calcd for C₁₁H₂₀O₂Na: 207.1361; 207.1358 (1 ppm).

Compound P7 (mixture of diastereoisomers): ¹H RMN δ (ppm): 0.86 (t, J=6.8 Hz, 3H, CH₂—CH₃); 0.96 and 0.97 (2d, J=6.8 Hz, 3H, CH—CH₃); 1.18 (s, 6H, C(OH)(CH₃)₂); 1.24-1.57 (m, 14H, 7CH₂); 2.05-2.18 (m, 1H, CH—CH₃); 4.01 (q, J=6.4 Hz, 1H, CHOH); 5.38 and 5.39 (2dd, J=15.6, 6.4 Hz, 1H, ═CHCHOH); 5.46 and 5.47 (2dd, J=15.6, 7.6 Hz, 1H, CH═CHCHOH). ¹³C RMN δ (ppm): 14.0; 20.6 (2); 21.9; 22.0; 22.6; 25.1; 25.2; 29.1; 29.2; 29.3; 31.7; 36.3; 36.4; 37.1; 37.3; 37.4; 43.8; 43.9; 71.0; 73.1; 73.2; 131.6; 137.5; 137.7. HRMS (ESI) calcd for C₁₆H₃₂O₂Na: 279.2300; found: 279.2300 (0 ppm).

Compound P8 (E isomer): RMN ¹H δ (ppm): 0.87 (t, J=6.8 Hz, 3H, CH₂—CH₃); 0.95 (d, J=6.8 Hz, 3H, CH—CH₃); 1.20 (s, 6H, C(OH)(CH₃)₂); 1.23-1.46 (m, 18H, 9CH₂); 1.93-2.00 (q, J=6.6 Hz, 2H, ═CH—CH₂); 2.05 (hept, J=6.8 Hz, 1H, CH—CH₃); 5.23 (ddt, J=15.2, 7.6, 1.2 Hz, 1H, CH—CH═CH); 5.35 (dt, J=15.2, 6.8 Hz, 1H, CH═CH—CH₂). ¹³C RMN δ (ppm): 14.1; 21.0; 22.1; 22.7; 29.1; 29.2; 29.3; 29.4; 29.7; 31.9; 32.6; 36.7; 37.6; 44.0; 71.1; 128.7; 136.1. HRMS (ESI) calcd for C₁₈H₃₆ONa: 291.26639; found: 291.2663 (0 ppm).

Compound P9 (E isomer): ¹H RMN δ (ppm): 0.95 (d, J=6.8 Hz, 3H, CH—CH₃); 1.19 (s, 6H, C(OH)(CH₃)₂); 1.23-1.65 (m, 16H, 8CH₂); 1.96 (q, J=6.8 Hz, 2H, ═CH—CH₂); 2.06 (hept, J=7.0 Hz, 1H, CH—CH₃); 2.29 (t, J=7.2 Hz, 2H, CH₂COOMe); 3.66 (s, 3H, OCH₃); 5.23 (ddt, J=15.2, 7.6, 1.2 Hz, 1H, CH—CH═CH), 5.33 (dtd, J=15.6, 6.4, 0.5, 1H, CH═CH—CH₂). ¹³C RMN δ (ppm): 21.0; 22.0; 24.9; 28.9; 29.1; 29.2; 29.6; 32.5; 34.1; 36.7; 37.6; 44.0; 51.4; 71.0; 128.6; 136.2; 174.3. HRMS (ESI) calcd for C₁₉H₃₆O₃Na: 335.25622; found 335.2566 (1 ppm).

Compound P10 (mixture of diastereoisomers): ¹H RMN δ (ppm): 0.95 and 0.96 (2d, J=6.4 Hz, 6H, CH—CH₃); 1.19 and 1.20 (2s, 12H, C(OH)(CH₃)₂); 1.20-1.48 (m, 12H, 6CH₂); 2.00-2.12 (m, 2H, CH—CH₃); 5.13-5.24 (m, 2H, CH═CH). ¹³C RMN δ (ppm): 21.1; 21.4; 21.9; 22.0; 29.1; 29.2; 29.5; 36.6; 37.0; 37.6; 37.7; 43.9; 71.0; 71.1; 134.5; 134.7. HRMS (ESI) calcd for C₁₈H₃₆O₂Na: 307.26075; found: 307.2613 (2 ppm).

Compound P11: ¹H RMN δ (ppm): 0.94 (t, J=7.6 Hz, 3H, CH₂—CH₃); 1.04 (d, J=6.8 Hz, 3H, CH—CH₃); 1.32-1.46 and 1.56-1.70 (m, 6H, 3CH₂); 1.58 and 1.68 (br s, 6H, C(CH₃)₂); 1.98 (q, 2H, J=7.2 Hz, CH₂—CH═); 2.31 (hept, J˜7 Hz, 1H, CH—CH═); 4.12 (t, J=6.8 Hz, 2H, CH₂O); 5.04-5.09 (m, 1H, CH═CMe₂); 5.77 (dd, J=15.6 Hz, 1.2, 1H, CH═CHCO); 6.86 (dd, J=15.6, 8.0 Hz, 1H, CH—CH═CH). ¹³C RMN, δ (ppm): 13.7; 17.7; 19.2; 19.3; 25.6; 25.7; 30.7; 36.0; 36.1; 64.1; 119.7; 124.0; 131.9; 154.4; 167.0. Compound P11′ selected value: ¹H RMN δ (ppm) 4.65 and 4.69 (2 br s, 2H, C═CH₂). HRMS (ESI) (mixture of P11 and P11′) calcd for C₁₅H₂₆O₂Na: 261.18305; found 261.1830 (0 ppm).

Compound P12: ¹H RMN δ (ppm): 0.94 (t, J=7.4 Hz, 3H, CH₂—CH₃); 1.06 (d, J=6.8 Hz, 3H, CH—CH₃); 1.35-1.68 (m, 6H, 3CH₂); 2.12-2.24 (m, 2H, CH₂); 2.28-2.39 (m, 1H, CH—CH=); 3.72 (s, 3H, CO₂CH₃); 4.13 (t, 2H, J=6.6 Hz, CH₂O); 5.79 (dd, J=15.6, 1.2 Hz, 1H, CH═CHCO₂Bu); 5.81 (dt, J=15.6, ˜1.5 Hz, 1H, CH═CHCO₂Me); 6.81 (dd, J=15.6, 8.2, 1H, CH—CH═CH); 6.92 (dt, J=15.6, 7.0, 1H, CH₂—CH═CH). ¹³C RMN, δ (ppm): 13.7; 19.2; 19.4; 29.8; 30.7; 34.1; 35.9; 51.4; 64.2; 120.4; 121.3; 148.6; 153.2; 166.8; 167.0. HRMS (ESI): m/z [M+Na]⁺ calcd for C₁₅H₂₄O₄Na: 291.1572; found: 291.1575 (1 ppm).

1. A process for obtaining a terpenoid derivative, the process comprising:
a metathesis of an olefin and a terpenoid, wherein the terpenoid has the following general formula: 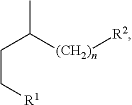
wherein R¹ is 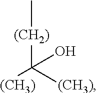
n is 0 and R² is 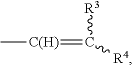
or
R¹ is 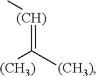
n is 0 and R² is 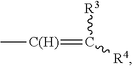
or
R¹ is 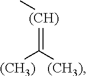
n is 1 and R² is —C(H)═O or —C(H₂)—OAc, and
wherein R³ and R⁴ are the same or different and are each independently hydrogen or alkyl, and
wherein the olefin has the following general formula: 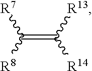
wherein R⁷, R⁸, R¹³ and R¹⁴ are the same or different and are each independently hydrogen, alkyl, halo, haloalkyl, alkenyl, alkynyl, cycloalkyl, aryl, arakyl, alkoxy, carbonyl, carboxyl, hydroxyl, amide, sulfonamide, or amine;
wherein when n=1, R⁷ and R⁸ are not both —CH₃; and
wherein when n=0, R⁷ and R⁸ together are different from R³ and R⁴ together.

2. A process for obtaining a terpenoid derivative, the process comprising the process according to claim 1 and a second metathesis of a second olefin and a terpenoid derivative obtained with the process of claim 1, wherein the second olefin has the following general formula: 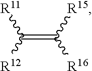
wherein R¹¹, R¹², R¹⁵ and R¹⁶ are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine;
wherein R¹¹ and R¹² are not both —CH₃; and
wherein R¹¹ and R¹² together are different from R³ and R⁴ together.

3. The process of claim 1, wherein the terpenoid has only one double bond.

4. The process of claim 1, wherein the terpenoid has at least two double bonds.

5. The process of claim 4, the process further comprising oxidizing at least one allylic carbon of the terpenoid prior to the olefin metathesis.

6. The process of claim 1, wherein R¹ is 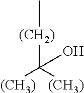
and the process further comprises a dehydration reaction after the olefin metathesis.

7. The process of claim 1, wherein the olefin cross-metathesis is a catalyzed olefin cross-metathesis.

8. The process of claim 7, wherein the catalyst is a ruthenium Hoveyda type catalyst.

9. The process of claim 8, wherein the ruthenium Hoveyda type catalyst has the general formula: 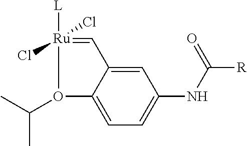
wherein and R is CF₃, CO₂Et, OiBu, C₆F₅ or C₁₅H₃₁, and
wherein L is

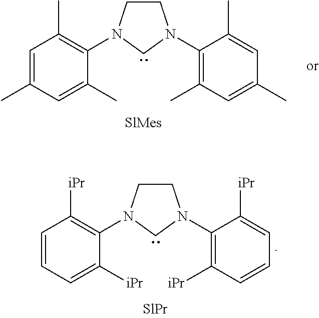

10. The process of claim 8, wherein the ruthenium Hoveyda type catalyst is 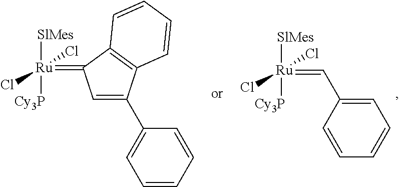
wherein SIMes is

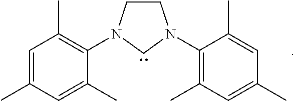

11. A terpenoid derivative prepared by the process according to claim 1.

12. A terpenoid derivative having the following general formula: 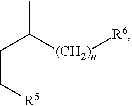
wherein R⁵ is 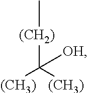
n is 0 and R⁶ is 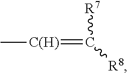
or
R⁵ is 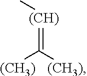
n is 0 and R⁶ is 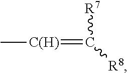
or
R⁵ is 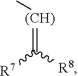
n is 0 and R⁶ is 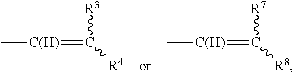
or
R⁵ is 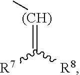
n is 1 and R⁶ is —C(H)═O or —C(H₂)—OAc,
wherein R³ and R⁴ are the same or different and are each independently hydrogen or alkyl,
wherein R⁷ and R⁸ are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine, and
wherein when R⁵ is 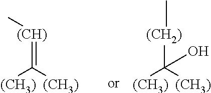
R⁷ and R⁸ are not both hydrogen, and
wherein when R⁵ is 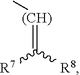
R⁷ and R⁸ are not both —CH₃.

13. A terpenoid derivative having the following general formula: 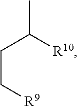
wherein R⁹ is 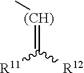
and R¹⁰ is 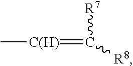
or
R⁹ is 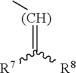
and R¹⁰ is 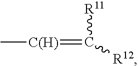
wherein R⁷, R⁸, R¹¹ and R¹² are the same or different and are each independently hydrogen, alkyl, halo, haloalkyl, alkenyl, alkynyl, cycloalkyl, aryl, aralkyl, alkoxy, carbonyl, carboxyl, hydroxyl, amide, sulfonamide, or amine,
wherein when R⁹ is 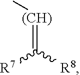
and when R¹¹ and R¹² are both hydrogen, R⁷ and R⁸ are not both —CH₃, and
wherein when R⁹ is 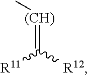
and when R⁷ and R⁸ are both hydrogen, R¹¹ and R¹² are not both —CH₃.

14. The terpenoid derivative of claim 13, wherein R⁷, R⁸, R¹¹, R¹² are the same or different and are each independently hydrogen, a lower alkyl, aryl, ketone, ester, ether, amide, or sulfonamide.

15. Use of a ruthenium Hoveyda type catalyst for the preparation of a terpenoid derivative by the catalyzed olefin cross-metathesis of a terpenoid with an olefin, the catalyst having the general formula: 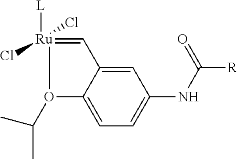
wherein and R is CF₃, CO₂Et, OiBu, C₆F₅ or C₁₅H₃₁, and
wherein L is 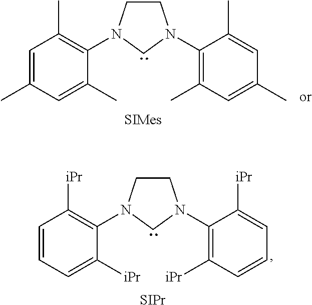
or having the general formula: 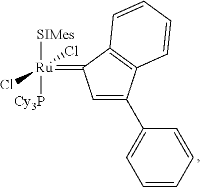
or having the general formula 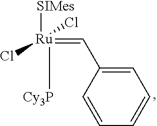
wherein SIMes is [chemical structure]
